(19) United States
(12) Patent Application Publication
(10) Pub. No.: US 2023/0212156 A1
(43) Pub. Date: Jul. 6, 2023

(54) METHOD FOR PRODUCING NITROFURANTOIN ANHYDRATE, AND PRODUCT THEREOF

(71) Applicant: FERMION OY, Espoo (FI)

(72) Inventors: Oskari KARJALAINEN, ESPOO (FI); Mikko LESKINEN, ESPOO (FI)

(73) Assignee: FERMION OY, Espoo (FI)

(21) Appl. No.: 17/924,826

(22) PCT Filed: May 11, 2021
(86) PCT No.: PCT/FI2021/050345

§ 371 Date: Nov. 11, 2022

(30) Foreign Application Priority Data
May 12, 2020 (FI) 20205474

(51) Int. Cl. C07D 405/12

(52) U.S. Cl. CPC C07D 405/12; C07B 2200/13

(57) ABSTRACT

The present disclosure relates to a novel process for production of crystalline particles of nitrofurantoin anhydrate, the crystalline particles of nitrofurantoin anhydrate being obtained by a process as disclosed, and pharmaceutical compositions as disclosed including crystalline particles of nitrofurantoin anhydrate.

### FIELD OF THE INVENTION

The present invention relates to a process for the production of nitrofurantoin anhydrate and to the products obtained by the process.

### BACKGROUND OF THE INVENTION

Nitrofurantoin or 1-[[(5-nitro-2-furanyl)methylene]amino]-2,4-imidazolidinedione is represented by the chemical formula (I) presented below.

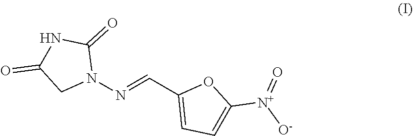 [SMILES: O=C1CN(/N=C/c2ccc([N+](=O)[O-])o2)C(=O)N1]

Nitrofurantoin is an antibiotic. Pharmacologically, nitrofurantoin is provided as either the anhydrate (macrocrystal form) or as the monohydrate. The present invention concerns a method for the production of crystalline particles of nitrofurantoin anhydrate, specifically by re-crystallisation of a source of nitrofurantoin.

Nitrofurantoin anhydrate is sparingly soluble in most organic solvents like alcohols, esters, ethers and hydrocarbons, with notable exceptions being polar aprotics like dimethylformamide (as used in e.g. CN 101450940). Dimethylformamide is included on the candidate list of substances of very high concern rendering its use in active pharmaceutical ingredient production undesirable. Due to the limitations presented by solubility, the control of the crystallization process to produce nitrofurantoin anhydrate with a desired particle size distribution is challenging. As an added difficulty, nitrofurantoin anhydrate is not stable as concentrated solution for long periods of time which is evident from the formation of black color in solution.

Nitrofurantoin anhydrate can alternatively be crystallised from various acids. For example, nitrofurantoin anhydrate has been crystallised from sulfuric acid and mixtures containing sulfuric acid (IN 201711010738) and from formic acid (Drug Dev. Ind. Pharm. 6(4), 379-398 (1980) and Int. J. Pharm. 55 (1989), 257-263). However, due to the poor solubility of nitrofurantoin, the crystallisation methods often include using high solvent volumes and result in low yield.

Further, many preparation methods for nitrofurantoin anhydrate have a problem in that nitrofurantoin is poorly soluble and has a tendency, on re-crystallisation, to form crystals of a large size which are unsuitable for direct pharmaceutical formulation.

Thus, it is desirable to provide a safe and practicable re-crystallisation method for the production of nitrofurantoin anhydrate. It is also desirable to provide a re-crystallisation method in that particle size distribution of nitrofurantoin anhydrate is easily controlled.

### SUMMARY OF THE INVENTION

The present invention provides a method for the preparation of crystalline particles of nitrofurantoin anhydrate, the method comprising the steps of:
(a) dissolving a first source of nitrofurantoin in a volume of a first solvent that is a C₁ to C₃ carboxylic acid so as to form a solution of nitrofurantoin;
(b) inducing crystallisation of nitrofurantoin to form crystalline particles of nitrofurantoin anhydrate by combining, in a container, the solution of step (a) and a volume of a mixture of water and a second solvent that is a C₁ to C₃ carboxylic acid, wherein the mixture of water and second solvent optionally further contains an amount of a second source of nitrofurantoin; and
(c) collecting the crystalline particles of nitrofurantoin anhydrate.

In an embodiment, the method of the present invention further comprises a step (d) of formulating the crystalline particles of nitrofurantoin anhydrate into a pharmaceutical composition.

The present invention also provides crystalline particles of nitrofurantoin anhydrate obtainable by the method of the invention.

The present invention also provides crystalline particles of nitrofurantoin anhydrate wherein the particles have an Air Jet Sieve (AJS) particle size distribution of:

>75 micron NLT 95%;

>180 micron NLT 50%; and

<425 micron NMT 10%;

wherein NLT indicates “not less than” and NMT indicates “not more than”.

In a particular embodiment, the present invention provides crystalline particles of nitrofurantoin anhydrate wherein the particles have an Air Jet Sieve (AJS) particle size distribution of:

>75 micron: about 99%;

>180 micron: about 84%; and

<425 micron: about 94%.

The present invention also provides crystalline particles of nitrofurantoin anhydrate wherein the particles have a laser light diffraction particle size distribution of:

Dv10: NLT 100 micron;

Dv50: NLT 240 micron; and

Dv90: NMT 670 micron;

wherein NLT indicates “not less than” and NMT indicates “not more than”.

The present invention also provides a pharmaceutical composition comprising the crystalline particles of nitrofurantoin anhydrate of the invention and optionally one or more pharmaceutically acceptable carriers.

The method of the present invention is advantageous in that it is environmentally friendly and uses solvents generally regarded as safe. For example, it does not involve the use of solvents such as dimethylformamide (DMF).

The method of the present invention is also advantageous in that it requires relatively small volumes of solvents.

The method of the present invention is also advantageous in that achieves generally good yields.

The method of the present invention is also advantageous in that the particle size distribution of the product crystalline particles of nitrofurantoin anhydrate is easily controlled.

The method of the present invention is also advantageous in that it allows for the production of crystalline particles of nitrofurantoin anhydrate having a relatively narrow particle size distribution. The method also allows for the production of crystalline particles of nitrofurantoin anhydrate having a desired particle size having good absorption and dissolution properties.

### BRIEF DESCRIPTION OF THE DRAWINGS

FIG. 1—Particle size distribution (laser light diffraction) of examples 2a, 2b and 2c.

FIG. 2—Particle size distribution (laser light diffraction) of example 6.

FIG. 3—Particle size distribution (laser light diffraction) of example 7.

FIG. 4—Particle size distribution (laser light diffraction) of comparison example 8.

### DETAILED DESCRIPTION OF THE INVENTION

### Method of the Invention

Method

As was described above, the present invention provides a method for the preparation of crystalline particles of nitrofurantoin anhydrate, the method comprising the steps of:
(a) dissolving a first source of nitrofurantoin in a volume of a first solvent that is a C₁ to C₃ carboxylic acid so as to form a solution of nitrofurantoin;
(b) inducing crystallisation of nitrofurantoin to form crystalline particles of nitrofurantoin anhydrate by combining, in a container, the solution of step (a) and a volume of a mixture of water and a second solvent that is a C₁ to C₃ carboxylic acid, wherein the mixture of water and second solvent optionally further contains an amount of a second source of nitrofurantoin; and
(c) collecting the crystalline particles of nitrofurantoin anhydrate.

Materials

In an embodiment of the present invention, the first solvent may be the same C₁ to C₃ carboxylic acid as the second solvent or may be a different C₁ to C₃ carboxylic acid to the second solvent. Thus, in one embodiment, the first solvent is formic acid and the second solvent is formic acid; or the first solvent is formic acid and the second solvent is acetic acid; or the first solvent is formic acid and the second solvent is propionic acid; or the first solvent is acetic acid and the second solvent is formic acid; or the first solvent is acetic acid and the second solvent is acetic acid; or the first solvent is acetic acid and the second solvent is propionic acid; or the first solvent is propionic acid and the second solvent is formic acid; or the first solvent is propionic acid and the second solvent is acetic acid; or the first solvent is propionic acid and the second solvent is propionic acid. In a preferred embodiment of the method of the present invention, the first solvent is the same C₁ to C₃ carboxylic acid as the second solvent. Thus, preferably, the first solvent is formic acid and the second solvent is formic acid; or the first solvent is acetic acid and the second solvent is acetic acid; or the first solvent is propionic acid and the second solvent is propionic acid. Most preferably, the first solvent and the second solvent are each formic acid.

In an embodiment of the method of the present invention, 600 to 1200 mL of the first solvent is used per 100 g of the first source of nitrofurantoin. Thus, the volume of the first solvent can be expressed as being from 6 to 12 volume/weight based on the weight of the first source of nitrofurantoin, and may preferably be from 7 to 11 volume/weight, further preferably 7.5 to 10.5 volume/weight, even further preferably 8 to 10 volume/weight, more preferably 8.5 to 9.5 volume/weight, even more preferably 8.7 to 9.3 volume/weight, and more preferably still 8.9 to 9.1 volume/weight. In a particularly preferred embodiment of the method of the present invention, the volume of the first solvent is about 9 volume/weight based on the weight of the first source of nitrofurantoin.

Suitably, the C₁ to C₃ carboxylic acid used as the first solvent has a concentration of >88% by weight, preferably >95%, more preferably >98%, even more preferably >99%, most preferably 100%. Thus, preferably, the formic acid used as the first solvent in the method of the invention has a concentration of >88% by weight, preferably >95%, more preferably >98%, even more preferably >99%, most preferably 100%.

In the method of the present invention, the first source of nitrofurantoin is suitably nitrofurantoin monohydrate or nitrofurantoin anhydrate, and may further be a mixture of nitrofurantoin monohydrate and nitrofurantoin anhydrate. In one embodiment, though, the first source of nitrofurantoin is nitrofurantoin monohydrate. In another embodiment, the first source of nitrofurantoin is nitrofurantoin anhydrate.

In an embodiment of the method of the present invention, 300 to 900 mL of the mixture of water and second solvent is used per 100 g of the first source of nitrofurantoin. Thus, the volume of the mixture of water and second solvent may be expressed as being from 3 to 9 volume/weight based on the weight of the first source of nitrofurantoin, and may preferably be from 4 to 9 volume/weight, further preferably 4 to 8 volume/weight, even further preferably 4 to 7 volume/weight, more preferably 4 to 6 volume/weight, even more preferably 4.5 to 5.5 volume/weight. In a particularly preferred embodiment of the method of the present invention, the volume of the mixture of water and second solvent is about 5 volume/weight based on the weight of the first source of nitrofurantoin.

In an embodiment of the method of the present invention, the mixture of water and second solvent is a 25:75-75:25 volume/volume mixture of water and second solvent; preferably a 30:70 to 70:30 volume/volume mixture of water and second solvent, more preferably a 35:65-65:35 volume/volume mixture of water and second solvent, further preferably a 40:60 to 60:40 volume/volume mixture of water and second solvent, more preferably a 45:55 to 55:45 volume/volume mixture of water and second solvent, even more preferably a 47:53 to 53:47 volume/volume mixture of water and second solvent, more preferably still a 49:51 to 51:49 volume/volume mixture of water and second solvent. In a particularly preferred embodiment of the method of the present invention, the mixture of water and second solvent is about a 50:50 volume/volume mixture of water and second solvent.

The skilled person will appreciate that any concentration source of C₁ to C₃ carboxylic acid may be employed in preparing the mixture of water and second solvent, provided that the water to second solvent ratios are met. Thus, for example, the skilled person would appreciate that a 50:50 volume/volume mixture of water and second solvent could be achieved by mixing equal parts of (i) pure water and (ii) a C₁ to C₃ carboxylic acid having a concentration of 100%, but could alternatively be achieved by the direct use of a source of C₁ to C₃ carboxylic acid having a concentration of 50% by volume (wherein the remainder of the source of C₁ to C₃ carboxylic acid is water). Thus, suitably, the source of the C₁ to C₃ carboxylic acid used in preparing the mixture of water and second solvent has a C₁ to C₃ carboxylic acid concentration (by volume) of 30% or greater, preferably 50% or greater, further preferably 70% or greater, even further preferably 85% or greater, more preferably 90% or greater, even more preferably 95% or greater, more preferably still 99% or greater, and most preferably 100%. Thus, suitably, the source of formic acid used in preparing the mixture of water and second solvent (wherein the second solvent is formic acid) has a formic acid concentration (by volume) of 30% or greater, preferably 50% or greater, further preferably 70% or greater, even further preferably 85% or greater, more preferably 90% or greater, even more preferably 95% or greater, more preferably still 99% or greater, and most preferably 100%. Particularly suitably, the source of formic acid used in preparing the mixture of water and second solvent (wherein the second solvent is formic acid) has a formic acid concentration (by weight) of 50% or greater, preferably 70% or greater, even further preferably 85% or greater, more preferably 90% or greater, even more preferably 95% or greater, more preferably still 99% or greater, and most preferably 100%.

If used, the second source of nitrofurantoin may be employed in any amount, but particularly in an amount suitable to provide seed crystals for the growth of particles of nitrofurantoin anhydrate. Thus, the second source of nitrofurantoin is in crystal form. Thus, in an embodiment of the method of the present invention, the amount of the second source of nitrofurantoin is from 0.01 to 20 weight % based on the weight of the first source of nitrofurantoin, preferably from 0.1 to 10 weight %, further preferably from 0.2 to 5 weight %, even more preferably from 0.3 to 3 weight %, even further preferably from 0.4 to 1 weight %. In a particularly preferred embodiment of the method of the present invention, the amount of the second source of nitrofurantoin is about 0.5 weight % based on the weight of the first source of nitrofurantoin.

Suitably, the second source of nitrofurantoin is nitrofurantoin monohydrate or nitrofurantoin anhydrate, and is most preferably nitrofurantoin anhydrate.

In an embodiment of the method of the present invention, following the combination of the solution of step (a) and the mixture of water and second solvent, a cumulative total volume of 900 to 2100 mL of water, first solvent and second solvent is present in the container of step (b) per 100 g of the first source of nitrofurantoin. Thus, following the combination of the solution of step (a) and the mixture of water and second solvent, the cumulative total volume of water, first solvent and second solvent present in the container of step (b) may be expressed as being from 9 to 21 volume/weight based on the weight of the first source of nitrofurantoin, and may preferably be from 10 to 20 volume/weight, further preferably 11.5 to 18 volume/weight, more preferably 12.5 to 17 volume/weight, and even more preferably 12.5 to 16 volume/weight. In a particularly preferred embodiment of the method of the present invention, following the combination of the solution of step (a) and the mixture of water and second solvent, the cumulative total volume of water, first solvent and second solvent present in the container of step (b) is about 13 volume/weight based on the weight of the first source of nitrofurantoin.

Processing Steps

In an embodiment, step (a) of the method of the present invention is carried out under an inert atmosphere. The inert atmosphere is preferably a nitrogen atmosphere or argon atmosphere, and is most preferably a nitrogen atmosphere.

Step (a) of the method of the present invention is carried out to completely dissolve the first source of nitrofurantoin in the volume of the first solvent (i.e. so as to form a clear solution of nitrofurantoin). Thus, in a preferred embodiment of the present invention, step (a) is carried out with mixing, preferably moderate to vigorous mixing (e.g. by stirring at a speed of from 50 revolutions per minute (RPM) to >800 RPM, optionally at >100 RPM, >200 RPM, >300 RPM, >400 RPM, >500 RPM, or >700 RPM. In one embodiment, the mixing is carried out at a speed of from 50 rpm to 400 rpm, preferably from 100 rpm to 300 rpm. In another preferred embodiment of the present invention, step (a) is carried out at a temperature of >30° C., preferably >50° C., more preferably >70° C., even more preferably >80° C., most preferably >85° C. In another preferred embodiment, step (a) is carried out at a temperature of <101° C., preferably <100° C., more preferably <97° C., even more preferably <95° C. Suitably, step (a) is carried out at a temperature of around 90° C.

In an embodiment of the method of the present invention, the solution of step (a) is preferably combined with the volume of the mixture of water and second solvent by addition of the solution of step (a) to the mixture of water and second solvent. The addition may be carried out by any means known to the person skilled in the art, but is suitably carried out feeding under pressure, preferably under nitrogen pressure, or using a pump.

In an embodiment of the method of the present invention, the mixture of water and second solvent is held at a temperature of from 35 to 45° C., preferably about 40° C., prior to combination with the solution of step (a).

In an embodiment, step (b) of the method of the present invention is carried out under an inert atmosphere. The inert atmosphere is preferably a nitrogen atmosphere or argon atmosphere, and is most preferably a nitrogen atmosphere.

In an embodiment of the method of the present invention, in step (b), the solution of step (a) is combined with the mixture of water and second solvent over a period of 5 minutes to one hour, preferably 10 minutes to 45 minutes, most preferably 15 minutes to 30 minutes.

In an embodiment of the present invention, in step (b), the solution of step (a) is combined with the mixture of water and second solvent with mixing, preferably moderate to vigorous mixing (e.g. by stirring at a speed of from 50 revolutions per minute (RPM) to >800 RPM, optionally at >100 RPM, >200 RPM, >300 RPM, >400 RPM, >500 RPM, or >700 RPM. In one embodiment, the mixing is carried out at a speed of from 50 rpm to 400 rpm, preferably from 100 rpm to 300 rpm. This turbulent mixing assists in directing the formed crystals away from the interface so as to avoid increasing the crystal particle size.

In an embodiment of the present invention, in step (b), the contents of the container during the combination of the solution of step (a) and the volume of the mixture of water and second solvent are held at a temperature of from 40 to 60° C., preferably from 45 to 55° C., most preferably from 45 to 50° C.

In another embodiment of the present invention, in step (b), the contents of the container following the combination of the solution of step (a) and the volume of the mixture of water and second solvent are adjusted to a temperature of from 50 to 60° C., preferably adjusted to a temperature of from 52 to 60° C., further preferably adjusted to a temperature of from 55 to 60° C., more preferably adjusted to a temperature of about 55° C., and are held at that temperature for a period of time, optionally with mixing. This step assists in “ageing” the crystals: agglomerates fall apart, small crystals dissolve, and larger crystals grow.

In a preferred embodiment, in step (b), the contents of the container during the combination of the solution of step (a) and the volume of the mixture of water and second solvent are held at a temperature of from 45 to 55° C., preferably from 45 to 50° C.; and the contents of the container following the combination of the solution of step (a) and the volume of the mixture of water and second solvent are adjusted to a temperature of from 52 to 60° C., preferably 55 to 60° C. (preferably adjusted to a temperature of about 55° C.), and are held at that temperature for a period of time, optionally with mixing.

In the above embodiments, suitably, the period of time may be up to 1 week, suitably up to 1 day, but is preferably from 1 minute to 5 hours, further preferably from 30 minutes to 4 hours, more preferably 1 hour to 3 hours, and is most preferably about 2 hours. Suitably, the mixing may be moderate to vigorous mixing (i.e. stirring at a speed of from 50 revolutions per minute (RPM) to >800 RPM, optionally at >100 RPM, >200 RPM, >300 RPM, >400 RPM, >500 RPM, or >700 RPM). In one embodiment, the mixing is carried out at a speed of from 50 rpm to 400 rpm, preferably from 100 rpm to 300 rpm.

In an embodiment of the present invention, following step (b), the contents of the container are cooled over a period of time, optionally with mixing. Preferably, the contents of the container are cooled to a temperature of from 10 to 30° C., preferably 15 to 25° C., more preferably from 17 to 23° C., more preferably still from 19 to 21° C., and is most preferably cooled to a temperature of about 20° C.

Suitably, the contents of the container are cooled over a period of from 10 minutes to 10 hours, preferably from 20 minutes to 8 hours, more preferably from 30 minutes to 6 hours, even more preferably from 1 hour to 5 hours, even further preferably from 2 hours to 4 hours, and most preferably the contents of the container are cooled over a period of about 3 hours. Suitably, the mixing may be moderate to vigorous mixing (i.e. stirring at a speed of from 50 revolutions per minute (RPM) to >800 RPM, optionally at >100 RPM, >200 RPM, >300 RPM, >400 RPM, >500 RPM, or >700 RPM). In one embodiment, the mixing is carried out at a speed of from 50 rpm to 400 rpm, preferably from 100 rpm to 300 rpm. This mixing assists in preventing mass from gathering at the bottom of the container.

Optionally, following the contents of the container being cooled, the contents of the container are mixed for a period of time. Suitably, the contents of the container are mixed for period of from 5 minutes to 10 hours, preferably from 10 minutes to 4 hours, more preferably from 15 minutes to 2 hours, even more preferably from 20 minutes to 1 hour, even further preferably from 25 minutes to 45 minutes, and most preferably the contents of the container are cooled over a period of about 30 minutes. Suitably, the mixing may be moderate to vigorous mixing (i.e. stirring at a speed of from 50 revolutions per minute (RPM) to >800 RPM, optionally at >100 RPM, >200 RPM, >300 RPM, >400 RPM, >500 RPM, or >700 RPM). In one embodiment, the mixing is carried out at a speed of from 50 rpm to 400 rpm, preferably from 100 rpm to 300 rpm.

In step (c) of the present invention, the crystalline particles of nitrofurantoin anhydrate may be collected by any means, but are suitably collected by filtration or centrifugation. Thus, in one embodiment, the crystalline particles of nitrofurantoin anhydrate are collected by filtration. In another embodiment, the crystalline particles of nitrofurantoin anhydrate are collected by centrifugation.

In a further embodiment of the method of the present invention, following step (c), the crystalline particles of nitrofurantoin anhydrate are washed. Any suitable solvent may be used to wash the crystalline particles of nitrofurantoin anhydrate. However, in a preferred embodiment, the crystalline particles of nitrofurantoin anhydrate are washed with one or more solvents selected from the group consisting of water; C₁-C₅ alcohols such as methanol, ethanol and isopropanol; and C₂-C₁₀ esters such as methyl acetate and ethyl acetate. Preferably, the crystalline particles of nitrofurantoin anhydrate are washed with an C₁-C₅ alcohol such as methanol, ethanol or isopropanol, and are most preferably washed with ethanol.

In a further preferred embodiment of the method of the present invention, the crystalline particles of nitrofurantoin anhydrate obtained in step (c) are dried. The particles may be dried by any means known in the art, but may suitably be dried under ambient conditions or under a vacuum, with or without heating.

In a preferred embodiment of the method of the present invention, the crystalline particles of nitrofurantoin anhydrate obtained in step (c) are de-clumped. This assists in homogenising the crystalline particles of nitrofurantoin anhydrate. The particles may be de-clumped by any means known in the art, but may suitably be de-clumped by passing through a coarse sieve (for example a 1 mm sieve) or by passing through a conical mill.

Post-Processing Steps

In an embodiment, the method of the present invention further comprises a step (d) of formulating the crystalline particles of nitrofurantoin anhydrate into a pharmaceutical composition.

Any pharmaceutical composition known to the persons skilled in the art is envisaged. Typically, however, the pharmaceutical composition will be suitable for oral administration, and will be in the form of a tablet, a gelatin capsule or an oral suspension comprising the crystalline particles of nitrofurantoin anhydrate, optionally with nitrofurantoin monohydrate, and one or more excipients. When the pharmaceutical composition is in the form a tablet or gelatin capsule, the excipients may suitably comprise one or more of:
(a) diluents, such as lactose, dextrose, sucrose, mannitol, sorbitol, cellulose and/or glycine;
(b) lubricants, such as silica, talc, stearic acid (and/or its magnesium or calcium salt) and/or polyethyleneglycol;
(c) binders, such as magnesium aluminium silicate, starch paste, gelatin, tragacanth, methylcellulose, sodium carboxymethylcellulose and/or polyvinylpyrrolidone;
(d) disintegrants, such as starches, agar, alginic acid (or its sodium salt) and/or effervescent mixtures;
(e) absorbents, colourants, flavours and/or sweeteners.

In a preferred embodiment, the pharmaceutical composition will comprise crystalline particles of nitrofurantoin anhydrate together with lactose monohydrate, maize starch and purified talc within a gelatin capsule.

In another preferred embodiment, the pharmaceutical composition will comprise crystalline particles of nitrofurantoin anhdrate together with lactose, maize starch, pregelatinised maize starch, sodium starch glycollate, magnesium stearate and purified water, within a tablet.

In another preferred embodiment, the pharmaceutical composition will comprise crystalline particles of nitrofurantoin anhydrate together with nitrofurantoin monohydrate and talc, corn starch, lactose carbopol, povidone, sugar and magnesium stearate within a gelatin capsule. The crystalline particles of nitrofurantoin anhydrate will preferably form 25% of the active ingredients of the composition, and the nitrofurantoin monohydrate will preferably form 75% of the active ingredients of the composition.

In a further preferred embodiment, the pharmaceutical composition will comprise crystalline particles of nitrofurantoin anhydrate together with glycerol, polysorbate-20, carbomer, saccharin sodium, methyl parahydroxybenzoate (E218), propyl parahydroxybenzoate (E216), sodium hydroxide, flavourings (such as Lemon Essence F31874 and Apricot Flavour F31191) and purified water, within an oral suspension.

Specifically Disclosed Embodiments

The present invention has the following preferred embodiments having one or more of the preferred features discussed above. However, for the avoidance of doubt, the inventors fully contemplate that one or more further features as discussed above may incorporated into each of the embodiments below.

Thus, in one preferred embodiment, the present invention provides a method for the preparation of crystalline particles of nitrofurantoin anhydrate, the method comprising the steps of:
(a) dissolving a first source of nitrofurantoin in a volume of a first solvent that is a C₁ to C₃ carboxylic acid so as to form a solution of nitrofurantoin;
(b) inducing crystallisation of nitrofurantoin to form crystalline particles of nitrofurantoin anhydrate by combining, in a container, the solution of step (a) and a volume of a mixture of water and a second solvent that is a C₁ to C₃ carboxylic acid, wherein the mixture of water and second solvent optionally further contains an amount of a second source of nitrofurantoin; and
(c) collecting the crystalline particles of nitrofurantoin anhydrate; wherein: (i) the first solvent and the second solvent are each formic acid.

In another preferred embodiment, the present invention provides a method for the preparation of crystalline particles of nitrofurantoin anhydrate, the method comprising the steps of:
(a) dissolving a first source of nitrofurantoin in a volume of a first solvent that is a C₁ to C₃ carboxylic acid so as to form a solution of nitrofurantoin;
(b) inducing crystallisation of nitrofurantoin to form crystalline particles of nitrofurantoin anhydrate by combining, in a container, the solution of step (a) and a volume of a mixture of water and a second solvent that is a C₁ to C₃ carboxylic acid, wherein the mixture of water and second solvent optionally further contains an amount of a second source of nitrofurantoin; and
(c) collecting the crystalline particles of nitrofurantoin anhydrate; wherein:
   (i) the first solvent and the second solvent are each formic acid; and
   (ii) the solution of step (a) is combined with the volume of a mixture of water and second solvent by addition of the solution of step (a) to the mixture of water and second solvent.

In a further preferred embodiment, the present invention provides a method for the preparation of crystalline particles of nitrofurantoin anhydrate, the method comprising the steps of:

(a) dissolving a first source of nitrofurantoin in a volume of a first solvent that is a C₁ to C₃ carboxylic acid so as to form a solution of nitrofurantoin;

(b) inducing crystallisation of nitrofurantoin to form crystalline particles of nitrofurantoin anhydrate by combining, in a container, the solution of step (a) and a volume of a mixture of water and a second solvent that is a C₁ to C₃ carboxylic acid, wherein the mixture of water and second solvent optionally further contains an amount of a second source of nitrofurantoin; and
(c) collecting the crystalline particles of nitrofurantoin anhydrate; wherein:
   (i) the first solvent and the second solvent are each formic acid; and
   (ii) the solution of step (a) is combined with the volume of a mixture of water and second solvent by addition of the solution of step (a) to the mixture of water and second solvent; and
   (iii) the volume of the first solvent is from 6 to 12 volume/weight, preferably 7.5 to 10.5 volume/weight, based on the weight of the first source of nitrofurantoin.

In a further preferred embodiment, the present invention provides a method for the preparation of crystalline particles of nitrofurantoin anhydrate, the method comprising the steps of:

(a) dissolving a first source of nitrofurantoin in a volume of a first solvent that is a C₁ to C₃ carboxylic acid so as to form a solution of nitrofurantoin;

(b) inducing crystallisation of nitrofurantoin to form crystalline particles of nitrofurantoin anhydrate by combining, in a container, the solution of step (a) and a volume of a mixture of water and a second solvent that is a C₁ to

C₃ carboxylic acid, wherein the mixture of water and second solvent optionally further contains an amount of a second source of nitrofurantoin; and
(c) collecting the crystalline particles of nitrofurantoin anhydrate; wherein:
   (i) the first solvent and the second solvent are each formic acid; and
   (ii) the solution of step (a) is combined with the volume of a mixture of water and second solvent by addition of the solution of step (a) to the mixture of water and second solvent; and
   (iii) the volume of the first solvent is from 6 to 12 volume/weight, preferably 7.5 to 10.5 volume/weight, based on the weight of the first source of nitrofurantoin; and
   (iv) the volume of the mixture of water and second solvent is from 3 to 9 volume/weight, preferably 4 to 9 volume/weight, based on the weight of the first source of nitrofurantoin.

In a further preferred embodiment, the present invention provides a method for the preparation of crystalline particles of nitrofurantoin anhydrate, the method comprising the steps of:
(a) dissolving a first source of nitrofurantoin in a volume of a first solvent that is a C₁ to C₃ carboxylic acid so as to form a solution of nitrofurantoin;
(b) inducing crystallisation of nitrofurantoin to form crystalline particles of nitrofurantoin anhydrate by combining, in a container, the solution of step (a) and a volume of a mixture of water and a second solvent that is a C₁ to C₃ carboxylic acid, wherein the mixture of water and second solvent optionally further contains an amount of a second source of nitrofurantoin; and
(c) collecting the crystalline particles of nitrofurantoin anhydrate; wherein:
   (i) the first solvent and the second solvent are each formic acid; and
   (ii) the solution of step (a) is combined with the volume of a mixture of water and second solvent by addition of the solution of step (a) to the mixture of water and second solvent; and
   (iii) the volume of the first solvent is from 6 to 12 volume/weight, preferably 7.5 to 10.5 volume/weight, based on the weight of the first source of nitrofurantoin; and
   (iv) the volume of the mixture of water and second solvent is from 3 to 9 volume/weight, preferably 4 to 9 volume/weight, based on the weight of the first source of nitrofurantoin; and
   (v) the mixture of the mixture of water and second solvent is a 25:75 to 75:25 volume/volume mixture of water and second solvent.

In a further preferred embodiment, the present invention provides a method for the preparation of crystalline particles of nitrofurantoin anhydrate, the method comprising the steps of:
(a) dissolving a first source of nitrofurantoin in a volume of a first solvent that is a C₁ to C₃ carboxylic acid so as to form a solution of nitrofurantoin;
(b) inducing crystallisation of nitrofurantoin to form crystalline particles of nitrofurantoin anhydrate by combining, in a container, the solution of step (a) and a volume of a mixture of water and a second solvent that is a C₁ to C₃ carboxylic acid, wherein the mixture of water and second solvent optionally further contains an amount of a second source of nitrofurantoin; and
(c) collecting the crystalline particles of nitrofurantoin anhydrate; wherein:
   (i) the first solvent and the second solvent are each formic acid; and
   (ii) the solution of step (a) is combined with the volume of a mixture of water and second solvent by addition of the solution of step (a) to the mixture of water and second solvent; and
   (iii) the volume of the first solvent is from 6 to 12 volume/weight, preferably 7.5 to 10.5 volume/weight, based on the weight of the first source of nitrofurantoin; and
   (iv) the volume of the mixture of water and second solvent is from 3 to 9 volume/weight, preferably 4 to 9 volume/weight, based on the weight of the first source of nitrofurantoin; and
   (v) the mixture of the mixture of water and second solvent is a 25:75 to 75:25 volume/volume mixture of water and second solvent; and
   (vi) in step (b), the contents of the container during the combination of the solution of step (a) and the volume of the mixture of water and second solvent are held at a temperature of from 45 to 55° C., preferably 45 to 50° C.

In a further preferred embodiment, the present invention provides a method for the preparation of crystalline particles of nitrofurantoin anhydrate, the method comprising the steps of:
(a) dissolving a first source of nitrofurantoin in a volume of a first solvent that is a C₁ to C₃ carboxylic acid so as to form a solution of nitrofurantoin;
(b) inducing crystallisation of nitrofurantoin to form crystalline particles of nitrofurantoin anhydrate by combining, in a container, the solution of step (a) and a volume of a mixture of water and a second solvent that is a C₁ to C₃ carboxylic acid, wherein the mixture of water and second solvent optionally further contains an amount of a second source of nitrofurantoin; and
(c) collecting the crystalline particles of nitrofurantoin anhydrate; wherein:
   (i) the first solvent and the second solvent are each formic acid; and
   (ii) the solution of step (a) is combined with the volume of a mixture of water and second solvent by addition of the solution of step (a) to the mixture of water and second solvent; and
   (iii) the volume of the first solvent is from 6 to 12 volume/weight, preferably 7.5 to 10.5 volume/weight, based on the weight of the first source of nitrofurantoin; and
   (iv) the volume of the mixture of water and second solvent is from 3 to 9 volume/weight, preferably 4 to 9 volume/weight, based on the weight of the first source of nitrofurantoin; and
   (v) the mixture of the mixture of water and second solvent is a 25:75 to 75:25 volume/volume mixture of water and second solvent; and
   (vi) in step (b), the contents of the container during the combination of the solution of step (a) and the volume of the mixture of water and second solvent are held at a temperature of from 45 to 55° C., preferably 45 to 50° C.; and
   (vii) in step (b), the contents of the container following the combination of the solution of step (a) and the volume of the mixture of water and second solvent are adjusted to a temperature of from 50 to 60° C. and are held at that temperature for a period of time.

In a further preferred embodiment, the present invention provides a method for the preparation of crystalline particles of nitrofurantoin anhydrate, the method comprising the steps of:
(a) dissolving a first source of nitrofurantoin in a volume of a first solvent that is a C₁ to C₃ carboxylic acid so as to form a solution of nitrofurantoin;
(b) inducing crystallisation of nitrofurantoin to form crystalline particles of nitrofurantoin anhydrate by combining, in a container, the solution of step (a) and a volume of a mixture of water and a second solvent that is a C₁ to C₃ carboxylic acid, wherein the mixture of water and second solvent optionally further contains an amount of a second source of nitrofurantoin; and
(c) collecting the crystalline particles of nitrofurantoin anhydrate; wherein:
   (i) the first solvent and the second solvent are each formic acid; and
   (ii) the solution of step (a) is combined with the volume of a mixture of water and second solvent by addition of the solution of step (a) to the mixture of water and second solvent; and
   (iii) the volume of the first solvent is from 6 to 12 volume/weight, preferably 7.5 to 10.5 volume/weight, based on the weight of the first source of nitrofurantoin; and
   (iv) the volume of the mixture of water and second solvent is from 3 to 9 volume/weight, preferably 4 to 9 volume/weight, based on the weight of the first source of nitrofurantoin; and
   (v) the mixture of the mixture of water and second solvent is a 25:75 to 75:25 volume/volume mixture of water and second solvent; and
   (vi) in step (b), the contents of the container during the combination of the solution of step (a) and the volume of the mixture of water and second solvent are held at a temperature of from 45 to 55° C., preferably 45 to 50° C.; and
   (vii) in step (b), the contents of the container following the combination of the solution of step (a) and the volume of the mixture of water and second solvent are adjusted to a temperature of from 50 to 60° C. and are held at that temperature for a period of time; and
   (viii) following step (b), the contents of the container are cooled.

In a further preferred embodiment, the present invention provides a method for the preparation of crystalline particles of nitrofurantoin anhydrate, the method comprising the steps of:
(a) dissolving a first source of nitrofurantoin in a volume of a first solvent that is a C₁ to C₃ carboxylic acid so as to form a solution of nitrofurantoin;
(b) inducing crystallisation of nitrofurantoin to form crystalline particles of nitrofurantoin anhydrate by combining, in a container, the solution of step (a) and a volume of a mixture of water and a second solvent that is a C₁ to C₃ carboxylic acid, wherein the mixture of water and second solvent further contains an amount of a second source of nitrofurantoin, wherein the second source of nitrofurantoin is nitrofurantoin anhydrate; and
(c) collecting the crystalline particles of nitrofurantoin anhydrate; wherein:
   (i) the first solvent and the second solvent are each formic acid; and
   (ii) the solution of step (a) is combined with the volume of a mixture of water and second solvent by addition of the solution of step (a) to the mixture of water and second solvent; and
   (iii) the volume of the first solvent is from 6 to 12 volume/weight, preferably 7.5 to 10.5 volume/weight, based on the weight of the first source of nitrofurantoin; and
   (iv) the volume of the mixture of water and second solvent is from 3 to 9 volume/weight, preferably 4 to 9 volume/weight, based on the weight of the first source of nitrofurantoin; and
   (v) the mixture of the mixture of water and second solvent is a 25:75 to 75:25 volume/volume mixture of water and second solvent; and
   (vi) in step (b), the contents of the container during the combination of the solution of step (a) and the volume of the mixture of water and second solvent are held at a temperature of from 45 to 55° C., preferably 45 to 50° C.; and
   (vii) in step (b), the contents of the container following the combination of the solution of step (a) and the volume of the mixture of water and second solvent are adjusted to a temperature of from 50 to 60° C. and are held at that temperature for a period of time; and
   (viii) following step (b), the contents of the container are cooled.

Particles of the Invention

As was described above, the present invention also provides crystalline particles of nitrofurantoin anhydrate obtained by or obtainable by the method of the invention.

As was also described above, the present invention also provides crystalline particles of nitrofurantoin anhydrate wherein the particles have an Air Jet Sieve (AJS) (mass weighted) particle size distribution of:

>75 micron NLT 95%;

>180 micron NLT 50%; and

>425 micron NMT 10%;

wherein NLT indicates “not less than” and NMT indicates “not more than”.

In a preferred embodiment, the particles of the present invention have an Air Jet Sieve (AJS) particle size distribution of:

>75 micron NLT 96%; preferably >75 micron NLT 97%; more preferably >75 micron NLT 98%; and/or

>180 micron NLT 55%, preferably >180 micron NLT 60%, more preferably >180 micron NLT 65%; and/or

>425 micron NMT 8%, preferably >425 micron NMT 7%, more preferably >425 micron NMT 6%.

In a particularly preferred embodiment, the particles of the present invention have an Air Jet Sieve (AJS) particle size distribution of:

>75 micron: about 99%;

>180 micron: about 84%; and

<425 micron: about 94%.

Air jet sieving is a method for determining the particle size (sieve fraction) and mass-weighted particle size distribution of a bulk mixture of particles. In general, the method for carrying out an air jet sieving procedure is as follows.

Together with sample material the sieve is placed on the unit and covered with a lid. A powerful industrial vacuum cleaner generates a strong jet of air which disperses the particles on the sieve through the slotted nozzle rotating below the sieve mesh. Thus the particles are dispersed with each rotation and are distributed over the complete sieve surface. The jet of air causes a continuous new orientation of the particles on the sieve surface. Particles with sizes smaller than the sieve apertures are sucked in by the vacuum cleaner. When using sieves of certain height (e.g. 25 mm height), the inflowing air causes the particles to impact on the lid, thereby helping to destroy agglomerates. The material remaining on the sieve can then be weighed, allowing the amount of the sample having a particle size greater than the aperture of the mesh to be calculated.

To calculate particle size distributions using an air jet sieve, two different methods can be used, as follows.

Standard method: the complete amount of material to be sieved is placed on the sieve with the finest mesh size. After sieving and weighing the fraction, the oversize is placed on the next largest sieve and sieved again. This procedure is continued until the complete sample is separated into fractions.

“Swiss Method”: the sample is first divided into the number of size classes to be determined and then each part is sieved individually with the corresponding sieve. This method can only provide reliable results if the sample division was carried out representatively to keep the particle size distribution in all part samples identical. The best results are obtained by rotary sample dividers, which divide the initial material into (e.g.) 6, 8 or 10 identical part samples.

In the present invention, it is preferred that an Alpine air jet sieve (preferably an Alpine A200LS air jet sieve) is used in the air jet sieving method. Preferably, the sample amount is representative of the sample, and has a mass of at least 10 g. Thus, in one embodiment, the sample amount is 10 g. In this or any other embodiment, sieving time is suitably 3 minutes.

The sieves for use in the air jet sieving method are woven metal sieves. The technical requirements of the test sieves are specified in ISO standard ISO 3310-1:2016 Test sieves—Technical requirements and testing—Part 1: Test sieves of metal wire cloth. The sieves are also according to standard ASTM E11.

The sieving is done according to the process described in Ph.Eur. 2.9.38 Particle-size distribution estimation by analytical sieving. Air jet sieving method is used to determine the mass distribution of particles.

As was also described above, the present invention also provides crystalline particles of nitrofurantoin anhydrate wherein the particles have a laser light diffraction (volume weighted) particle size distribution of:

Dv10: NLT 100 micron;

Dv50: NLT 240 micron; and

Dv90: NMT 670 micron;

wherein NLT indicates “not less than” and NMT indicates “not more than”.

Laser light diffraction is a method for determining the volume-weighted particle size distribution of a bulk mixture of particles. In general, to obtain such a particle size distribution, a sample of interest is illuminated by laser light of a given wavelength. The technique relies upon the fact that the particles will scatter light when exposed to electromagnetic radiation. The resulting scattering pattern can be measured electronically and then deconvulated mathematically to infer a particle size distribution.

Dv10, Dv50 and Dv90 describe the diameter in gm where ten, fifty or ninety percent of the particle distribution respectively, has a smaller particle size than the specified diameter value. Dv10, Dv50 and Dv90 are based on volumetric distribution and are determined by assuming that the particles have a geometric shape equivalent to a sphere.

In the present invention, it is preferred that a Beckman Coulter laser diffraction particle size analyser (preferably a Beckman Coulter LS 230 laser diffraction particle size analyser equipped with Micro-volume Module) is used in the laser light diffraction method. Preferably, the optical model used is the Fraunhofer model of laser diffraction. Preferably, the sample to be analysed is representative of the bulk product, and has a mass of around 100 mg. Preferably, the sample to be analysed is suspended in a mixture of glycerol and water. Preferably, the mixture is around 1:1 mixture of glycerol and water. The particle size analysis is done according to the process described in Ph.Eur. 2.9.31 (Particle size analysis by laser light diffraction) and according to ISO 13320-1 (1999) (Particle size analysis—Laser scattering methods—Part 1: General principles).

Pharmaceutical Composition of the Invention

As was described above, the present invention further provides a pharmaceutical composition comprising crystalline particles of nitrofurantoin anhydrate according to the present invention and optionally one or more pharmaceutically acceptable carriers.

Any pharmaceutical composition known to the persons skilled in the art is envisaged. Typically, however, the pharmaceutical composition will be suitable for oral administration, and will preferably be in the form of a tablet, a gelatin capsule or an oral suspension comprising the crystalline particles of nitrofurantoin anhydrate, optionally with nitrofurantoin monohydrate, and one or more excipients. When the pharmaceutical composition is in the form a tablet or gelatin capsule, the excipients may suitably comprise one or more of:
(a) diluents, such as lactose, dextrose, sucrose, mannitol, sorbitol, cellulose and/or glycine;
(b) lubricants, such as silica, talc, stearic acid (and/or its magnesium or calcium salt) and/or polyethyleneglycol;
(c) binders, such as magnesium aluminium silicate, starch paste, gelatin, tragacanth, methylcellulose, sodium carboxymethylcellulose and/or polyvinylpyrrolidone;
(d) disintegrants, such as starches, agar, alginic acid (or its sodium salt) and/or effervescent mixtures;
(e) absorbents, colourants, flavours and/or sweeteners.

In a preferred embodiment, the pharmaceutical composition will comprise crystalline particles of nitrofurantoin anhydrate together with lactose monohydrate, maize starch and purified talc within a gelatin capsule.

In another preferred embodiment, the pharmaceutical composition will comprise crystalline particles of nitrofurantoin anhydrate together with lactose, maize starch, pregelatinised maize starch, sodium starch glycollate, magnesium stearate and purified water, within a tablet.

In another preferred embodiment, the pharmaceutical composition will comprise crystalline particles of nitrofurantoin anhydrate together with nitrofurantoin monohydrate and talc, corn starch, lactose carbopol, povidone, sugar and magnesium stearate within a gelatin capsule. The particles of nitrofurantoin anhydrate will preferably form 25% of the active ingredients of the composition, and the nitrofurantoin monohydrate will preferably form 75% of the active ingredients of the composition.

In a further preferred embodiment, the pharmaceutical composition will comprise crystalline particles of nitrofurantoin anhydrate together with glycerol, polysorbate-20, carbomer, saccharin sodium, methyl parahydroxybenzoate (E218), propyl parahydroxybenzoate (E216), sodium hydroxide, flavourings (such as Lemon Essence F31874 and Apricot Flavour F31191) and purified water, within an oral suspension.

Therapeutic Indications

Further disclosed is a method for treating and/or preventing acute or recurrent, uncomplicated lower urinary tract infections or pyelitis, wherein the method comprises administering the crystalline particles of nitrofurantoin anhydrate of the invention or the pharmaceutical composition of the invention to a patient in need thereof.

Further disclosed are crystalline particles of nitrofurantoin anhydrate of the invention or a pharmaceutical composition of the invention for use in a method of treating and/or preventing acute or recurrent, uncomplicated lower urinary tract infections or pyelitis, wherein the method comprises administering the crystalline particles of nitrofurantoin anhydrate of the invention or the pharmaceutical composition of the invention to a patient in need thereof.

Further disclosed is the use of the crystalline particles of nitrofurantoin anhydrate of the invention or the pharmaceutical composition of the invention in the manufacture of a medicament for use in a method of treating and/or preventing acute or recurrent, uncomplicated lower urinary tract infections or pyelitis, wherein the method comprises administering the crystalline particles of nitrofurantoin anhydrate of the invention or the pharmaceutical composition of the invention to a patient in need thereof.

The uncomplicated lower urinary tract infections or pyelitis may either be spontaneous or follow surgical procedures.

The lower urinary tract infections may specifically be lower urinary tract infections due to susceptible strains of Escherichia coli, enterococci, staphylococci, Citrobacter, Klebsiella and Enterobacter.

The patient in need thereof may be any mammal, but is preferably a human. The human may be an adult, child or infant. Where the human is an infant, it is preferred that the infant is >3 months old.

For adults, the above methods may comprise administering the crystalline particles of nitrofurantoin anhydrate of the invention or the pharmaceutical composition of the invention in a 50 mg dosage form or a 100 mg dosage form with respect to the active ingredient(s). In one embodiment, the 50 mg dosage form will be administered four times daily, preferably for seven days. In a further embodiment, the 100 mg dosage form will be administered four times daily, preferably for seven days. In a further embodiment, the 50 mg dosage form will be administered once daily for an extended period of time, such as a period of time greater than one week, preferably greater than one month, more preferably greater than one year. In another embodiment, the 100 mg dosage form will be administered once daily for an extended period of time, such as a period of time greater than one week, preferably greater than one month, more preferably greater than one year. In another embodiment, the 50 mg dosage form will be administered four times daily for the duration of a procedure (such as a surgical procedure), and four times daily for three days thereafter.

For children and infants, the above methods may comprise administering the crystalline particles of nitrofurantoin anhydrate of the invention or the pharmaceutical composition of the invention in an amount of from 1 mg/kg to 3 mg/kg with respect to the active ingredient(s). In one embodiment, the method may comprise administering the crystalline particles of nitrofurantoin anhydrate of the invention or the pharmaceutical composition of the invention in an amount of 1 mg/kg with respect to the active ingredient(s), once daily. In another embodiment, the method may comprise administering the crystalline particles of nitrofurantoin anhydrate of the invention or the pharmaceutical composition of the invention in an amount of 3 mg/kg/day with respect to the active ingredient(s), wherein the 3 mg/kg amount is provided in four divided doses, preferably for seven days.

The present invention is further illustrated with the following non-limiting examples.

### EXAMPLES

### Example 1

To a reactor (reactor 1) under nitrogen blanket was charged formic acid (100 wt %, 800 mL) followed by nitrofurantoin monohydrate (100 g). The mixture was heated to 90° C. under mixing at 150 rpm, thus producing a clear solution.

To another reactor (reactor 2) under nitrogen blanket was added formic acid (100 wt %, 250 mL), water (250 mL) and about 0.5 g of nitrofurantoin anhydrate as seeds. The contents of the reactor were brought to 40° C. The contents of reactor 1 were fed to reactor 2 using nitrogen pressure over about 15-30 minutes while maintaining mixing at 150 rpm and keeping the reactor 2 temperature 45-52° C. After the addition the temperature of reactor 2 was raised to 55° C. and the suspension was stirred for 2 h prior to cooling to 20° C. over 3 h. The product was collected by filtration and washed with absolute ethanol (80 mL, 20° C.) (yield 65.6 g (70.5%)). After drying, the product was passed through a 1 mm sieve to delump the material.

The particle size distribution of the crystalline particles of nitrofurantoin anhydrate was measured using Alpine A200LS air jet sieve as described in example 10.

Particle size distribution (Alpine AJS):

>75 μm: 99%

>180 μm:76%<

425 μm: 96%

Thus, the present invention advantageously provides particles of nitrofurantoin anhydrate having a narrow particle size distribution and desired particle size for good dissolution and absorption rate. As may be seen, the method of present invention is also beneficial in that the method is relatively fast, allows the use of relatively small volumes of solvent, and also allows the production of particles of nitrofurantoin anhydrate in a good yield.

### Example 2

Nitrofurantoin monohydrate (100 g) was dissolved in reactor 1 at about 97° C. in formic acid (98 wt %, 8 vol, 800 mL) under nitrogen atmosphere. The solution from reactor 1 was added steadily to reactor 2 containing preheated (40° C.) and seeded mixture of formic acid (98 wt %, 2.5 vol, 250 mL) and water (2.5 vol, 250 mL). The addition was done using a pump over about 20-30 minutes while maintaining mixing at 250 rpm and keeping the reactor 2 at a temperature of about 45-48° C. After addition the mixture was first heated to 60° C. and stirred for 2 hours. Before the collection of the precipitate, the mixture was cooled to 20° C. over three hours and stirred about for 30 minutes. The solid was filtrated at about 20° C., washed with ethanol and finally dried under reduced pressure at 50° C. to yield crystalline particles of nitrofurantoin anhydrate (yield of experiment 2a, 2b and 2c, respectively: 71%, 73% and 73%). After drying, the product was passed through a 1 mm sieve to delump the material.

The experiment was done in three replicates (2a, 2b, 2c) using the above conditions, except that in experiment 2b, 75 g of nitrofurantoin monohydrate was dissolved in 600 ml of formic acid (98%), and the solution from reactor 1 was added to reactor 2 containing 188 mL formic acid (98%) and 188 mL of water. Also, in experiments 2b and 2c, the reactor 2 was kept at a temperature of about 47° C. during the addition of solution from reactor 1. Experiment 2a was seeded with 0.5 g (0.5 wt %) nitrofurantoin monohydrate and experiments 2b and 2c were seeded with 0.5 g nitrofurantoin anhydrate.

The particle size distribution of the crystalline particles of nitrofurantoin anhydrate was measured using Alpine A200LS air jet sieve as described in example 10.

Particle size distribution (Alpine AJS) of experiment 2a, 2b and 2c, respectively:

>75 μm: 98%, 98% and 98%

>180 μm: 72%, 67% and 70%<

425 μm: 95%, 98% and 95%

All the three replicate experiments yielded crystalline particles of nitrofurantoin anhydrate having a narrow particle size distribution and desired particle size acceptable for pharmaceutical formulation. The particle size distribution was easily controlled. As may be seen, the method is also beneficial in that the method is relatively fast, allows the use of relatively small volumes of solvent, and also allows the production of particles of nitrofurantoin anhydrate in good yield.

The particle size distribution of the crystalline particles of nitrofurantoin anhydrate of the three replicate experiments 2a, 2b and 2c was also determined by laser light diffraction according to the process described in Ph.Eur. 2.9.31 (Particle size analysis by laser light diffraction) and according to ISO 13320-1 (1999) (Particle size analysis—Laser scattering methods—Part 1: General principles). The determination was carried out by using Beckman Coulter LS 230 laser diffraction particle size analyzer equipped with Micro-volume Module. The optical model used was Fraunhofer model of laser diffraction. The particles were suspended in a mixture of glycerol and water (1:1). Sample size in analysis was 100 mg.

The measured Dv10, Dv50 and Dv90 values of the crystalline particles of experiments 2a, 2b and 2c, respectively, are shown below:

Dv10: 126 μm, 163 μm and 118 μm

Dv50: 264 μm, 348 μm and 252 μm

Dv90: 448 μm, 667 μm and 466 μm

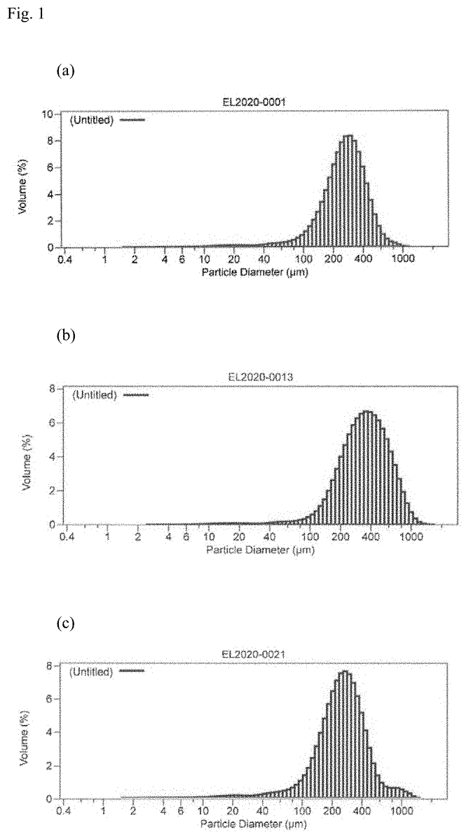

FIGS. 1a-1c respectively show the particle size distribution of crystalline particles of nitrofurantoin anhydrate of experiments 2a, 2b and 2c as analysed by laser light diffraction. Based on the figures, the crystalline particles have a narrow particle size distribution.

### Example 3

Nitrofurantoin monohydrate (100 g) was dissolved in reactor 1 at about 98° C. in formic acid (98 wt %, 10 vol, 1000 mL) under nitrogen atmosphere. The solution from reactor 1 was added steadily to reactor 2 containing preheated (40° C.) and seeded mixture of formic acid (98 wt %, 2.5 vol, 250 mL) and water (2.5 vol, 250 mL). The addition was done using a pump over about 40 minutes while maintaining mixing at 250 rpm and keeping the reactor 2 at a temperature of 47-48° C. After addition the mixture was first heated to 60° C. and stirred for 2 hours. Before the collection of the precipitate the mixture was cooled to 20° C. over three hours and stirred about for 30 minutes. The solid was filtrated at about 20° C., washed with ethanol and finally dried under reduced pressure at 50° C. to yield crystalline particles of nitrofurantoin anhydrate (yield 65%). After drying, the product was passed through a 1 mm sieve to delump the material.

The particle size distribution of the crystalline particles of nitrofurantoin anhydrate was measured using Alpine A200LS air jet sieve as described in example 10.

Particle size distribution (Alpine AJS):

>75 μm: 97%

>180 μm: 68%<

425 μm: 98%

The experiment yielded crystalline particles of nitrofurantoin anhydrate having a narrow particle size distribution and desired particle size acceptable for pharmaceutical formulation. The particle size distribution was easily controlled. As may be seen, the method is also beneficial in that the method is relatively fast, allows the use of relatively small volumes of solvent, and also allows the production of particles of nitrofurantoin anhydrate in good yield.

### Example 4

Nitrofurantoin monohydrate (100 g) was dissolved in reactor 1 at about 95° C. in formic acid (98 wt %, 9 vol, 900 mL) under nitrogen atmosphere. The solution from reactor 1 was added steadily to reactor 2 containing preheated (40° C.) and seeded mixture of formic acid (98 wt %, 4.5 vol, 450 mL) and water (2.5 vol, 250 mL). The addition was done using a pump over about 40 minutes while maintaining mixing at 250 rpm and keeping the reactor 2 at a temperature of about 47° C. After addition the mixture was first heated to 60° C. and stirred for 2 hours. Before the collection of the precipitate the mixture was cooled to 20° C. over three hours and stirred about for 30 minutes. The solid was filtrated at about 20° C., washed with ethanol and finally dried under reduced pressure at 50° C. to yield crystalline particles of nitrofurantoin anhydrate (yield 62%). After drying, the product was passed through a 1 mm sieve to delump the material.

The particle size distribution of the crystalline particles of nitrofurantoin anhydrate was measured using Alpine A200LS air jet sieve as described in example 10.

Particle size distribution (Alpine AJS):

>75 μm: 99%

>180 μm: 80%<

425 μm: 98%

The experiment yielded crystalline particles of nitrofurantoin anhydrate having a narrow particle size distribution and desired particle size acceptable for pharmaceutical formulation. The particle size distribution was easily controlled. As may be seen, the method is also beneficial in that the method is relatively fast, allows the use of relatively small volumes of solvent, and also allows the production of particles of nitrofurantoin anhydrate in good yield.

### Example 5

Three experiments (5a, 5b, 5c) were done as follows. Nitrofurantoin monohydrate (100 g) was dissolved in reactor 1 at about 98° C. in formic acid (98 wt %, 9 vol, 900 mL) under nitrogen atmosphere. The solution from reactor 1 was added steadily to reactor 2 containing preheated (40° C.) and seeded (0.5 g nitrofurantoin anhydrate) mixture of formic acid and water. The amount of formic acid (98 wt %) in reactor 2 before addition of solution from reactor 1 was 3.5 vol, 1.5 vol and 5.5 vol, and the amount of water was 1.5 vol, 3.5 vol and 3.2 vol for experiments 5a, 5b and 5c, respectfully. The addition was done using a pump over about 30 minutes while maintaining mixing at 250 rpm and keeping the reactor 2 at a temperature of about 47° C. After addition the mixture was first heated to 60° C. and stirred for 2 hours. Before the collection of the precipitate the mixture was cooled to 20° C. over three hours and stirred about for 30 minutes. The solid was filtrated at about 20° C., washed with ethanol and finally dried under reduced pressure at 50° C. to yield crystalline particles of nitrofurantoin anhydrate (yield of experiment 5a, 5b and 5c, respectively: 61%, 77%, 61%). After drying, the product was passed through a 1 mm sieve to delump the material.

The particle size distribution of the crystalline particles of nitrofurantoin anhydrate was measured using Alpine A200LS air jet sieve as described in example 10.

Particle size distribution (Alpine AJS) of experiment 5a, 5b and 5c, respectively:

>75 μm: 96%, 95%, 93%

>180 μm: 51%, 52%, 51%<

<425 μm: 97%, 98%, 99%

All the three experiments yielded crystalline particles of nitrofurantoin anhydrate having a narrow particle size distribution and desired particle size acceptable for pharmaceutical formulation. The particle size distribution was easily controlled. As may be seen, the method is also beneficial in that the method is relatively fast, allows the use of relatively small volumes of solvent, and also allows the production of particles of nitrofurantoin anhydrate in good yield.

### Example 6

Nitrofurantoin monohydrate (100 g) was dissolved in reactor 1 at about 98° C. in formic acid (98 wt %, 7 vol, 700 mL) under nitrogen atmosphere. The solution from reactor 1 was added steadily to reactor 2 containing preheated (40° C.) and seeded (0.5 g (nitrofurantoin anhydrate 0.5 wt %)) mixture of formic acid (98 wt %, 1.5 vol, 150 mL) and water (1.5 vol, 150 mL). The addition was done using a pump over about 30 minutes while maintaining mixing at 250 rpm and keeping the reactor 2 at a temperature of about 47° C.

After addition the mixture was first heated to 60° C. and stirred for 2 hours. Before the collection of the precipitate the mixture was cooled to 20° C. over three hours and stirred about for 30 minutes. The solid was filtrated at about 20° C., washed with ethanol and finally dried under reduced pressure at 50° C. to yield crystalline particles of nitrofurantoin anhydrate (yield 77%). After drying, the product was passed through a lmm sieve to delump the material.

The particle size distribution of the crystalline particles of nitrofurantoin anhydrate was measured using Alpine A200LS air jet sieve as described in example 10.

Particle size distribution (Alpine AJS):

>75 μm: 87%

>180 μm: 10%<

<425 μm: 98%

The particle size of the nitrofurantoin anhydrate prepared in this experiment was smaller than in example 2.

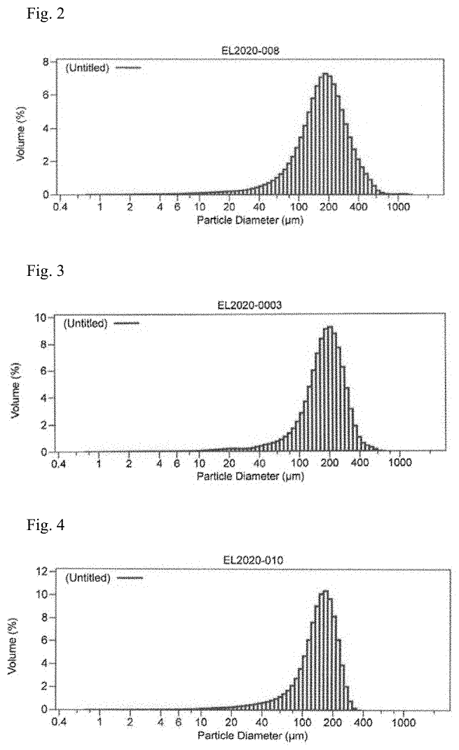

The particle size distribution of the crystalline particles of nitrofurantoin anhydrate was also determined by laser light diffraction according to the process described in Ph.Eur. 2.9.31 (Particle size analysis by laser light diffraction) and according to ISO 13320-1 (1999) (Particle size analysis—Laser scattering methods—Part 1: General principles). The determination was carried out by using Beckman Coulter LS 230 laser diffraction particle size analyzer equipped with Micro-volume Module. The optical model used was Fraunhofer model of laser diffraction. The particles were suspended in a mixture of glycerol and water (1:1). Sample size in analysis was 100 mg. FIG. 2 shows particle size distribution of crystalline particles of nitrofurantoin anhydrate of example 6 as analysed by laser light diffraction. Based on the figure, the size of the crystalline particles was smaller than in example 2.

### Example 7

Nitrofurantoin monohydrate (100 g) was dissolved in reactor 1 at about 98° C. in formic acid (98 wt %, 7 vol, 700 mL) under nitrogen atmosphere. The solution from reactor 1 was added steadily to reactor 2 containing preheated (40° C.) and seeded mixture of formic acid (98 wt %, 3.5 vol, 350 mL) and water (1.5 vol, 150 mL). The addition was done using nitrogen pressure over about 40 minutes while maintaining mixing at 250 rpm and keeping the reactor 2 at a temperature of about 47° C. After addition the mixture was first heated to 60° C. and stirred for 2 hours. Before the collection of the precipitate the mixture was cooled to 20° C. over three hours and stirred about for 30 minutes. The solid was filtrated at about 20° C., washed with ethanol and finally dried under reduced pressure at 50° C. to yield crystalline particles of nitrofurantoin anhydrate (yield 69%). After drying, the product was passed through a 1 mm sieve to delump the material.

The particle size distribution of the crystalline particles of nitrofurantoin anhydrate was measured using Alpine A200LS air jet sieve as described in example 10.

Particle size distribution (Alpine AJS):

>75 μm: 94%

>180 μm: 36%<

<425 μm: 99%

The particle size of the nitrofurantoin anhydrate prepared in this experiment was smaller than in example 2.

The particle size distribution of the crystalline particles of nitrofurantoin anhydrate was also determined by laser light diffraction according to the process described in Ph.Eur. 2.9.31 (Particle size analysis by laser light diffraction) and according to ISO 13320-1 (1999) (Particle size analysis—Laser scattering methods—Part 1: General principles). The determination was carried out by using Beckman Coulter LS 230 laser diffraction particle size analyzer equipped with Micro-volume Module. The optical model used was Fraunhofer model of laser diffraction. The particles were suspended in a mixture of glycerol and water (1:1). Sample size in analysis was 100 mg. FIG. 3 shows particle size distribution of crystalline particles of nitrofurantoin anhydrate of example 7 as analysed by laser light diffraction. Based on the figure, the size of the crystalline particles was smaller than in example 2.

### Comparison example 8

Nitrofurantoin was crystallized by the method of Garti N. and Tibika F., Drug Dev. Ind. Pharm., 1980, 6(4) 379-398.

To a reactor inerted with nitrogen was added 1500 mL of a solution containing a 4:1 mixture (weight:weight) of formic acid (98 wt %) and water, followed by nitrofurantoin monohydrate (35 g). The mixture was heated to about 40° C. to produce a clear solution.

The solution thus produced was cooled to 27° C. over 7 hours and stirred for 9.5 hours. The product was collected by filtration and washed with water (100 mL) and dried under vacuum at 50° C. Yield of crystalline particles of nitrofurantoin anhydrate was about 19%. After drying, the product was passed through a 1 mm sieve to delump the material.

The particle size distribution of the crystalline particles of nitrofurantoin anhydrate could not be measured using air jet sieve method since the amount of product was too small for sieving.

Instead, the particle size distribution of the crystalline particles of nitrofurantoin anhydrate was determined by laser light diffraction according to the process described in Ph.Eur. 2.9.31 (Particle size analysis by laser light diffraction) and according to ISO 13320-1 (1999) (Particle size analysis—Laser scattering methods—Part 1: General principles). The determination was carried out by using Beckman Coulter LS 230 laser diffraction particle size analyzer equipped with Micro-volume Module. The optical model used was Fraunhofer model of laser diffraction. The particles were suspended in a mixture of glycerol and water (1:1). Sample size in analysis was 100 mg. FIG. 4 shows particle size distribution of crystalline particles of nitrofurantoin anhydrate of comparison example 8 as analysed by laser light diffraction. Based on the figure, the size of the crystalline particles was smaller than in example 2. Also, the yield was small, and the process required large volume of solvent.

Comparison Example 9. Standard Crystallisation by Precipitation from a Solution Using Antisolvent

To a reactor inerted with nitrogen was added formic acid (85 wt %, 1350 mL) followed by nitrofurantoin monohydrate (150 g). The mixture was heated to about 95° C. to produce a clear solution. The temperature was then adjusted to 90° C. and the mixture was seeded.

Water (600 mL) was added over 1 h using a pump. The suspension thus produced was cooled to 20° C. over 4 h. The product was collected by filtration and washed with water (150 mL) and dried under vacuum at 50° C. Yield of crystalline particles of nitrofurantoin anhydrate was 118.7 g (85%). After drying, the product was passed through a 1 mm sieve to delump the material.

The particle size distribution of the crystalline particles of nitrofurantoin anhydrate was measured using Alpine A200LS air jet sieve as described in example 10.

Particle size distribution (Alpine AJS):

>75 μm: 99%

>180 μm: 89%

<425 μm: 27%

The crystalline particles of nitrofurantoin anhydrate prepared by the above method were too large and hence unsuitable for direct pharmaceutical formulation.

### Example 10. Measurement of Particle Size Distribution by Air Jet Sieve Method

Particle size distribution of crystalline particles of nitrofurantoin anhydrate was determined using Alpine A200LS air jet sieve. Sieving was done using woven metal sieves according to ISO standard ISO 3310-1:2016 (Test sieves—Technical requirements and testing—Part 1: Test sieves of metal wire cloth). The sieving was done using a single sieve at a time using the appropriate sieve size (75 gm, 180 gm, 425 gm). The sieving was done according to the process described in Ph.Eur. 2.9.38 (Particle-size distribution estimation by analytical sieving).

A container containing crystalline particles of nitrofurantoin anhydrate was inverted by rotating several times in order to ensure a homogenous sample. The sieve was tared. 10 grams of the particle mixture was weighed on the sieve. The sieve was placed on air jet sieve unit and covered with a lid. The vacuum cleaner was switched on, thereby creating a vacuum inside the sieving chamber. The sieving under vacuum was continued for 3 minutes. The finest particles were pulled downwards trough the sieve and the coarse particles remained on the sieve. At the end of the sieving process, the sieve was weighed and the mass of particles remained on the sieve was determined. The sieving process was done as described above using one sieve at a time until the particle size distribution was determined.

1. A method for preparation of crystalline particles of nitrofurantoin anhydrate, the method comprising:
(a) dissolving a first source of nitrofurantoin in a volume of a first solvent that is a C₁ to C₃ carboxylic acid so as to form a solution of nitrofurantoin;
(b) inducing crystallisation of nitrofurantoin to form crystalline particles of nitrofurantoin anhydrate by combining, in a container, the solution of step (a) and a volume of a mixture of water and a second solvent that is a C₁ to C₃ carboxylic acid; and
(c) collecting the crystalline particles of nitrofurantoin anhydrate.

2. The method according to claim 1, wherein the first solvent is a same C₁ to C₃ carboxylic acid as the second solvent.

3. The method according to claim 2, wherein the first solvent and the second solvent are each formic acid.

4. The method according to claim 1, wherein the volume of the first solvent is from 6 to 12 volume/weight based on a weight of the first source of nitrofurantoin, and/or 7 to 11 volume/weight, further and/or 7.5 to 10.5 volume/weight, and/or 8 to 10 volume/weight, and/or 8.5 to 9.5 volume/weight, and/or 8.7 to 9.3 volume/weight, and/or 8.9 to 9.1 volume/weight.

5. The method according to claim 4, wherein the volume of the first solvent is about 9 volume/weight based on the weight of the first source of nitrofurantoin.

6. The method according to claim 1, wherein the volume of the mixture of water and second solvent is from 3 to 9 volume/weight based on the weight of the first source of nitrofurantoin, and/or 4 to 9 volume/weight, and/or 4 to 8 volume/weight, and/or 4 to 7 volume/weight, and/or 4 to 6 volume/weight, and/or 4.5 to 5.5 volume/weight.

7. The method according to claim 6, wherein the volume of the mixture of water and second solvent is about 5 volume/weight based on the weight of the first source of nitrofurantoin.

8. The method according to claim 1, wherein the mixture of water and second solvent is a 25:75-75:25 volume/volume mixture of water and second solvent, and/or a 30:70 to 70:30 volume/volume mixture of water and second solvent, and/or a 35:65-65:35 volume/volume mixture of water and second solvent, and/or a 40:60 to 60:40 volume/volume mixture of water and second solvent, and/or a 45:55 to 55:45 volume/volume mixture of water and second solvent, and/or a 47:53 to 53:47 volume/volume mixture of water and second solvent, and/or a 49:51 to 51:49 volume/volume mixture of water and second solvent.

9. The method according to claim 8, wherein the mixture of water and second solvent is about a 50:50 volume/volume mixture of water and second solvent.

10. The method according to claim 1, wherein step (a) is carried out with mixing and/or at a temperature of >30° C., and/or >50° C., and/or >70° C., and/or >80° C., and/or >85° C.

11. The method according to claim 1, wherein the solution of step (a) is combined with the volume of a mixture of water and second solvent by addition of the solution of step (a) to the mixture of water and second solvent.

12. The method according to claim 1, wherein the mixture of water and second solvent is held at a temperature of from 35 to 45° C., and/or about 40° C., prior to combination with the solution of step (a).

13. The method according to claim 1, wherein, in step (b), the solution of step (a) is combined with the mixture of water and second solvent over a period of 5 minutes to one hour, and/or 10 minutes to 45 minutes, and/or 15 minutes to 30 minutes.

14. The method according to claim 1, wherein, in step (b), contents of the container during combination of the solution of step (a) and the volume of the mixture of water and second solvent are held at a temperature of from 40 to 60° C., and/or from 45 to 55° C., and/or from 45 to 50° C.

15. The method according to claim 1, wherein, in step (b), contents of the container following combination of the solution of step (a) and the volume of the mixture of water and second solvent are adjusted to a temperature of from 50 to 60° C., and/or adjusted to a temperature of from 52 to 60° C., and/or adjusted to a temperature of 55 to 60° C., and/or adjusted to a temperature of about 55° C., and are held at that temperature for a period of time.

16. The method according to claim 1, wherein, following step (b), contents of the container are cooled.

17. The method according to claim 16, wherein contents of the container are cooled to a temperature of from 10 to 30° C., and/or 15 to 25° C., and/or from 17 to 23° C., and/or from 19 to 21° C., and/or cooled to a temperature of about 20° C.

18. The method according to claim 16, wherein contents of the container are cooled over a period of from 10 minutes to 10 hours, and/or from 20 minutes to 8 hours, and/or from 30 minutes to 6 hours, and/or from 1 hour to 5 hours, and/or from 2 hours to 4 hours, and and/or the contents of the container are cooled over a period of about 3 hours.

19. The method according to claim 1, wherein, in step (c), the crystalline particles of nitrofurantoin anhydrate are collected by filtration or centrifugation.

20. The method of claim 1, wherein, following step (c), the crystalline particles of nitrofurantoin anhydrate are washed.

21. The method of claim 20, wherein the crystalline particles of nitrofurantoin anhydrate are washed with one or more solvents selected from the group consisting of water; C₁-C₅ alcohols, methanol, ethanol and isopropanol; C₂-C₁₀ esters, methyl acetate and ethyl acetate.

22. The method of claim 21, wherein particles of nitrofurantoin anhydrate are washed with C₁-C₅ alcohols, methanol, ethanol and/or isopropanol.

23. The method claim 1, wherein the first source of nitrofurantoin is nitrofurantoin monohydrate or nitrofurantoin anhydrate.

24. The method of claim 1, wherein the second source of nitrofurantoin is nitrofurantoin monohydrate or nitrofurantoin anhydrate, or nitrofurantoin anhydrate.

25. The method of claim 1, wherein the method comprises:
(d) formulating the crystalline particles of nitrofurantoin anhydrate into a pharmaceutical composition.

26. Crystalline particles of nitrofurantoin anhydrate obtained by a method according to claim 1.

27. Crystalline particles of nitrofurantoin anhydrate, wherein the particles have an Air Jet Sieve (AJS) particle size distribution of:
>75 micron NLT 95%;
>180 micron NLT 50%; and
>425 micron NMT 10%;
wherein NLT indicates “not less than” and NMT indicates “not more than”.

28. The crystalline particles of claim 27, wherein the particles have an Air Jet Sieve (AJS) particle size distribution of:
>75 micron: about 99%;
>180 micron: about 84%; and
<425 micron: about 94%.

29. Crystalline particles of nitrofurantoin anhydrate, wherein the particles have a laser light diffraction particle size distribution of:
Dv10: NLT 100 micron;
Dv50: NLT 240 micron; and
Dv90: NMT 670 micron;
wherein NLT indicates “not less than” and NMT indicates “not more than”.

30. A pharmaceutical composition comprising:
crystalline particles of nitrofurantoin anhydrate obtained according to claim 26.

31. The method according to claim 1, wherein the mixture of water and second solvent contains an amount of a second source of nitrofurantoin.

32. The pharmaceutical composition according to claim 30, comprising:
one or more pharmaceutically acceptable carriers.
